(19) United States
(12) Patent Application Publication
(10) Pub. No.: US 2020/0206257 A1
(43) Pub. Date: Jul. 2, 2020

(54) HOMOGENEOUS POLYSACCHARIDE ESP-B4, DERIVED FROM HERBA EPHEDRA, PREPARATION METHOD AND MEDICAL USE THEREOF

(71) Applicants: Haixue KUANG, Harbin (CN); Qiuhong WANG, Guangzhou (CN)

(72) Inventors: Haixue KUANG, Harbin (CN); Qiuhong WANG, Guangzhou (CN); Yonggang XIA, Harbin (CN)

(21) Appl. No.: 16/724,046

(22) Filed: Dec. 20, 2019

(30) Foreign Application Priority Data
Dec. 26, 2018 (CN) 201811599071.2

(51) Int. Cl. A61K 31/715; A61P 11/00; A61K 36/17; A61P 31/16; A61K 9/20; A61K 9/48; A61K 9/19; A61K 9/00; A61P 31/04

(52) U.S. Cl. CPC A61K 31/715; A61P 11/00; A61K 36/17; A61P 31/16; A61K 9/20; A61K 2236/53; A61K 9/19; A61K 9/0019; A61P 31/04; A61K 2236/39; A61K 2236/331; A61K 9/4808

(57) ABSTRACT

Provided is a homogeneous polysaccharide ESP-B4, method for producing the same and use thereof. The ESP-B4 is a polysaccharide monomer, which is isolated from the traditional Chinese medicine Ephedra sinica Stapf, Ephedra intermedia Schrenk et C. A. Mey, or Ephedra equisetina Bge. HPSEC-ELSD shows a single symmetrical peak. The chemical construction of ESP-B4 is an acidic heterosaccharide. It is composed of xylose, arabinose, glucose, rhamnose, mannose, galactose, glucuronic acid and galacturonic acid with a molar ratio of 1.0:4.5:1.0:2.0:5.5:1.5:50. The molecular weight of ESP-B4 is 2.37×10⁷ Da, and the content of galacturonic acid was 75.2%. The application of ESP-B4 is significant activity against infectious acute lung injury, respiratory distress syndrome, asthma, pneumonia, trachea and bronchitis. Therefore, it can be used for severe infectious acute lung injury caused by SARS virus, influenza a virus, avian influenza virus. Meanwhile, ESP-B4 has few side effects, simple preparation method, and it is suitable for industrial production.

### CROSS REFERENCE TO RELATED APPLICATIONS

This application claims the priority of Chinese Patent Application No. 201811599071.2, filed on Dec. 26, 2018, and the disclosures of which are hereby incorporated by reference.

### FIELD

The present disclosure relates to the field of medicine, in particular to a polysaccharide ESP-B4 from Ephedra sinica, preparation method and medical use thereof.

### BACKGROUND

Acute lung injury (ALI) can deteriorate to acute respiratory distress syndrome (ARDS). It is characterized by rapid respiratory failure and severe hypoxia of arterial blood which is not easy to be treated by oxygen transfusion, leading to multiple organ failure with a high fatality rate. And it is a critical disease with high morbidity and mortality in clinic. ALI/ARDS mortality rate is as high as 45%-50%. If accompanied by Sepsis, the mortality rate will increase high to 90%. However, if accompanied by multiple organ dysfunctions, the mortality rate will further increase. Unfortunately, the mortality rate will be 100% if accompanied by more than four organ dysfunction. The clinical manifestations of major infectious diseases such as SARS, influenza A and avian influenza are acute lung injury. In addition, respiratory diseases such as asthma, acute and chronic pneumonia, chronic bronchitis and chronic obstructive pulmonary disease can also lead to ALI/ARDS with the decline of environmental quality and the frequent occurrence of haze. Simple anti-inflammatory and anti-pathogenic treatment is difficult to change the occurrence and development of lung injury. There is a lack of effective therapeutic drugs in clinic to treat ALI. At present, antibiotics are applied in large quantities to fight against pathogenic bacteria, and hormones are used to fight against inflammation and alleviate lung injury with serious side effects. Therefore, the research and development of Anti-ALI and other respiratory inflammation drugs are urgently needed. The up-regulation of interleukin-8 (IL-8) may play an important role at the pathogenesis of ALI, which acts as a chemotactic cytokine in the lung to recruit the neutrophils from the blood vessel, and activate the neutrophils to release more cytokines to cause tissue damage. The patients of ALI may produce autoantibodies of IL-8, which in combination with IL-8 in local tissue, activates the corresponding signaling pathway, and induces the neutrophils and promotes the formation of inflammation.

Traditional Chinese medicine has unique advantages in treating lung injury. Its mechanism is not directly against pathogenic bacteria, but to reduce the occurrence of lung injury caused by autoimmunity by inhibiting the body's over-immune response to pathogenic bacteria antigen. This is a unique mechanism of action, which has been proved by long-term clinical practice of traditional Chinese medicine.

Ephedra sinica belongs to the lungs and kidney meridians. It has the functions of promoting lung, relieving asthma and promoting water, which is a famous Chinese medicine for treating lung diseases. It has been used for the treatment of pulmonary diseases such as chest tightness, cough and asthma since ancient times, and has a long history of application. Modern research erroneously believes that alkaloids such as ephedrine and pseudoephedrine are the effective active ingredients of Ephedra sinica. However, in the classical Chinese medicine literature “Treatise on Febrile Diseases”, Ephedra sinica is recorded as “boiling Ephedra sinica with nine liters of water firstly, reducing it by two liters, then removing foam and taking all kinds of medicines”. It has been identified that the decoction foam of Ephedra sinica contains a large amount of alkaloids, and it is pointed out that “Otherwise it would be irritating”, that means “Not removing ephedrine have the side effect of central excitation”. After activity tracking research, we innovatively found that the real effective substance basis of Ephedra sinica is polysaccharide components, and obtained the most content and most active polysaccharide monomer from Ephedra sinica, namely ESP-B4 by further separation and purification methods. The effect of ESP-B4 on respiratory system is different from the specific effect of western antibiotics and anti-inflammatory drugs. The common mechanism of ESP-B4 is related to the immunosuppressive activity, i.e. to achieve pharmacodynamics effect by inhibiting the excessive immune injury of the body. It is a unique mechanism for the role of active polysaccharides of traditional Chinese medicine.

### SUMMARY

The purpose of the invention is to provide a method for obtaining a homogeneous polysaccharide ESP-B4, and to provide an ESP-B4 preparation and a composite preparation for the treatment of respiratory diseases such as acute lung injury (ALI), respiratory distress syndrome, asthma, pneumonia, trachea and bronchitis.

On the one hand, the present invention provides a polysaccharide monomer, namely homogeneous polysaccharide ESP-B4, to treat respiratory diseases, such as acute lung injury, respiratory distress syndrome, asthma, pneumonia, trachea and bronchitis.

Preferably, the polysaccharide ESP-B4 is derived from the dry herbaceous stems of Ephedra sinica Stapf, Ephedra intermedia Schrenk et C. A. Mey, or Ephedra equisetina Bge.

Wherein, the content of ESP-B4 in raw materials of Ephedra sinica was 0.9-1.2% (W/W). Purity judgment by High Performance Size Exclusion Chromatography (HPSEC) or High Performance Capillary Electrophoresis (HPCE) both showed a single symmetrical peak, and that means ESP-B4 belongs to homogeneous polysaccharide. ESP-B4 consists of xylose, arabinose, glucose, rhamnose, mannose, galactose, glucuronic acid and galacturonic acid with molar ratio of 1.0:4.5:1.0:2.0:1.0:5.5:1.5:50, and the molar percentage of galacturonic acid was 75.2%. The molecular weight of ESP-B4 was about 2.37×10⁷ Da.

Preferably, ALI or respiratory distress syndrome is selected from respiratory diseases caused by infectious acute lung injury and its deterioration.

More preferably, the infectious ALI selected from the SARS virus, influenza A virus, avian flu virus etc., which causes major contagious ALI, and Escherichia coli, Pseudomonas aeruginosa, Pneumococcus, Staphylococcus aureus, Klebsiella pneumoniae, influenza virus, Chlamydia, Mycoplasma, which causes asthma, bronchitis, pneumonia.

The polysaccharide ESP-B4 was prepared according to the following steps:

(1) Water extraction and alcohol precipitation method was used to prepare total polysaccharide of Ephedra sinica.

(2) Series chromatography of Anion and Cation resin column was used to separate total polysaccharides.

(3) Furthermore, a homogeneous polysaccharide ESP-B4 was prepared by separation of cellulose and sepharose chromatography column by ion exchange, adsorption and molecular sieve mechanism.

The optimal step (1): extract 2-3 times with 5-10 times of hot water, and the ethanol concentration of alcohol precipitation is 75%-87%.

The optimal step (2): Amberlite FPC3500 and IRA-401 are preferred as the Anion and Cation resin fillers in series chromatography of Anion and Cation resin column.

The optimal step (3): The Cellulose column filler is Cellulose DE-52 or DEAE-Sepharose F.F.

The fillers used in step (2) and (3) are not limited to the above selection, and more fillers can be used, but the purity of the obtained homogeneous polysaccharide ESP-B4 should be more than 85%.

The invention adopts HPCE to determine the purity of ESP-B4, and the optimum conditions are as follows: 100 mM H₃BO₃—KOH (pH 10) as buffer solution, and detection wavelength is Uv 254 nm, quartz capillary column 48.5 cm×501 m with effective length 40 cm.

The invention adopts 1-phenyl-3-methyl-5-pyrazolinone (PMP) pre-column derivation HPCE method to determine monosaccharide composition of ESP-B4, and the optimization steps are as follows:

(1) Take various control monosaccharide (Mannose, GlcUA, GalUA, Rhamnose, Glucose, Galactose, Arabinose and xylose) and make them into water solution with a concentration of 1 mM with double distilled water to prepare standard monosaccharide control stock solution and mixed monosaccharide control solution.

(2) Weigh 100 mg of ESP-B4 sample, put it into a grinded reaction bottle, add 3 ml of 0.1M trifluoroacetic acid (TFA) to dissolve it, shake it fully to make it completely dissolved, and then close it, hydrolyze it in a 100° C. water bath for 1 h. The reaction solution was evaporated to dryness under decompression concentration, and methanol was added to the reaction solution, repetitive operation for 4 times to remove the residual trifluoroacetic acid. The hydrolyzed sample was dissolved in a small amount of water and then dialyzed (Mw 3500 Da) in distilled water for 48 hours. The solution in the dialysis bag is concentrated under reduced pressure, then precipitated by alcohol to obtain the precipitated part and the supernatant part. The secondary polysaccharide (a) was obtained by freeze-drying of the precipitation part, the secondary polysaccharide (b) was obtained by freeze-drying of the supernatant part, and the liquid outside the dialysis bag was freeze-dried, which was recorded as (c). ESP-B4 and its secondary polysaccharide components (a), (b) and (c) were carried out complete acid hydrolysis respectively. Weigh 20 mg of ESP-B4, (a), (b) and (c) sample respectively, put them into tubes with stopper, each add 2.0 mL of 2M H₂SO₄ solution. The sample solutions were hydrolyzed at 110° C. for 6 h after sealing. Neutralize to pH 7.0 with 4 M sodium hydroxide aqueous solution, dilute to 5.0 ml with purified water, after centrifugate, the supernatant for derivatization. It can help to understand the monosaccharide composition of ESP-B4 and their linkage.

(3) Derived mark: add 200 μL standard monosaccharide reference substance, mixed monosaccharide reference substance, ESP-B4 and hydrolyzate respectively to 100 μL PMP (0.5M methanol solution) and 100 μL sodium hydroxide solution (0.3 M) in centrifuge tube, heating reaction at 70° C. in water bath for 30 min, and then add 100 μL hydrochloric acid solution (0.3 M) to neutralize respectively. Equal volume isoamyl acetate was used for extraction, and the supernatant was discarded by 10 minutes centrifugation. Then isoamyl acetate was used for extraction once again, equal volume chloroform CHCl₃ was added. After vibration and 10 minutes centrifugation, the chloroform phase was discarded to obtain the upper water phase, dilute the water phase to 0.5 ml, after 0.45 μM microporous membrane filtrating for HPCE.

(4) Working conditions of capillary electrophoresis: 35 mmol/L borax, pH=10.02 as electrolyte buffer, anode 0.5 psi pressure injection, detection wavelength 254 nm, separation voltage +20 kV, column temperature 25° C. Then monosaccharide composition of polysaccharide ESP-B4 was determined as: xylose, arabinose, glucose, rhamnose, mannose, galactose, glucuronic acid, and galacturonic acid at a molar ratio of 1.0:4.5:1.0:2.0:5.5:1.5:50. Wherein, the content of galacturonic acid was 75.2%.

This invention adopts HPSEC-ELSD method to determine the molecular weight of homogeneous polysaccharide ESP-B4. Optimized conditions were as follows: Waters HPLC system (2996 pump, Alltech ELSD2000, Shodex sugar ks-805+protective column KS-G, mobile phase was ultrapure water, flow rate was 0.5 mL/min). Standard Dextran T10, Dextran T40, Dextran T70, Dextran T500 and Dextran T2000 solutions were prepared with the mobile phase to 5 mg/mL concentration before use, and the injection volume is 10 μL.

The standard curve was made taking log value of molecular weight as abscissa (x axis) and retention time RT as the ordinate (y axis).

The regression equation of the standard curve was obtained as y=−2.2303x+31.68, R²=0.9936. ESP-B4 was prepared using the same method. The molecular weight 2.37×10⁷ Da of ESP-B4 was calculated by the standard curve regression equation.

On the one hand, this invention provides a pharmaceutical composition or formulation, which contains homogeneous polysaccharide ESP-B4 and pharmaceutically acceptable carriers for medicinal purpose against ALI, respiratory distress syndrome, asthma, pneumonia, trachea and bronchitis.

### Beneficial Effects of this Invention

Pharmacological experiments showed that this invention homogeneous polysaccharide ESP-B4 has excellent activity of resistant respiratory system disease: infectious lung damage, respiratory distress syndrome, asthma, pneumonia, trachea and bronchitis, which could be used for the preparation of drug combination or formula to treat severe acute respiratory syndrome, such as SARS virus, influenza virus, avian influenza virus caused major contagious ALI as well as the lung injury, asthma, pneumonia, trachea and bronchitis caused by common infections such as Escherichia coli, Pseudomonas aeromonas, pneumococci, influenza virus, Chlamydia, and Mycoplasma, etc. This invention is a unique advantage of traditional Chinese medicine verified by long-term practice, and it has few side effects, and preparation method is simple and suitable for industrial production.

On the other hand, the present invention provides a pharmaceutical composition or formula possessing anti-acute lung injury, anti-respiratory distress syndrome, anti-asthma, anti-pneumonia, anti-trachea and anti-bronchitis activities, using ESP-B4 as the main active ingredient and pharmaceutically acceptable carrier. Preferably, ESP-B4 is the only active ingredient in the pharmaceutical composition.

The pharmaceutical composition or formula possessing anti-acute lung injury, anti-respiratory distress syndrome, anti-asthma, anti-pneumonia, anti-trachea and anti-bronchitis activities is prepared into any suitable clinical preparation by conventional preparation method. The pharmaceutical composition is in the form of an oral preparation or a parenteral preparation. The oral preparation said above is in the form of a tablet, a granule, a capsule, an oral liquid or a pill formulation. The parenteral preparation said above is in the form of injection or infusion. The capsule said above is in the form of a hard capsule or a soft capsule. The pill said above is in the form of a dripping pill. The injection said above is in the form of injection, freeze-dried powder injection, water injection, etc. The infusion said above is in the form of large infusion.

Preferably, the pharmaceutically acceptable excipients are in the form of diluents, adhesives, disintegrating agents, lubricants, flavoring agents, coating agents, gelatin capsule shells, skeleton materials, solvents, freeze-drying protectors, osmotic pressure regulators, etc.

The content of ESP-B4 in the pharmaceutical composition said above accounted for 1-99% of the total amount of the pharmaceutical composition, and the content of the ESP-B4 accounted for 15-40% of the pharmaceutical composition preferably.

The extraction and preparation process of the polysaccharide ESP-B4 of the invention being suitable for industrial production, provides sufficient raw materials for the follow-up study of ESP-B4, and broadens the application scope of the polysaccharide ESP-B4.

### BRIEF DESCRIPTION OF DRAWINGS

In order to more clearly illustrate the examples of the present disclosure or the technical solutions in the prior art, the drawings used in the examples or the prior art will be briefly described below. Obviously, the drawings in the following description are only the examples of the present application, and those skilled in the art can obtain other drawings according to the provided drawings without any creative work.

FIG. 1 is a plot elution curve with optical density value of ESP-B4 used in Example 1.

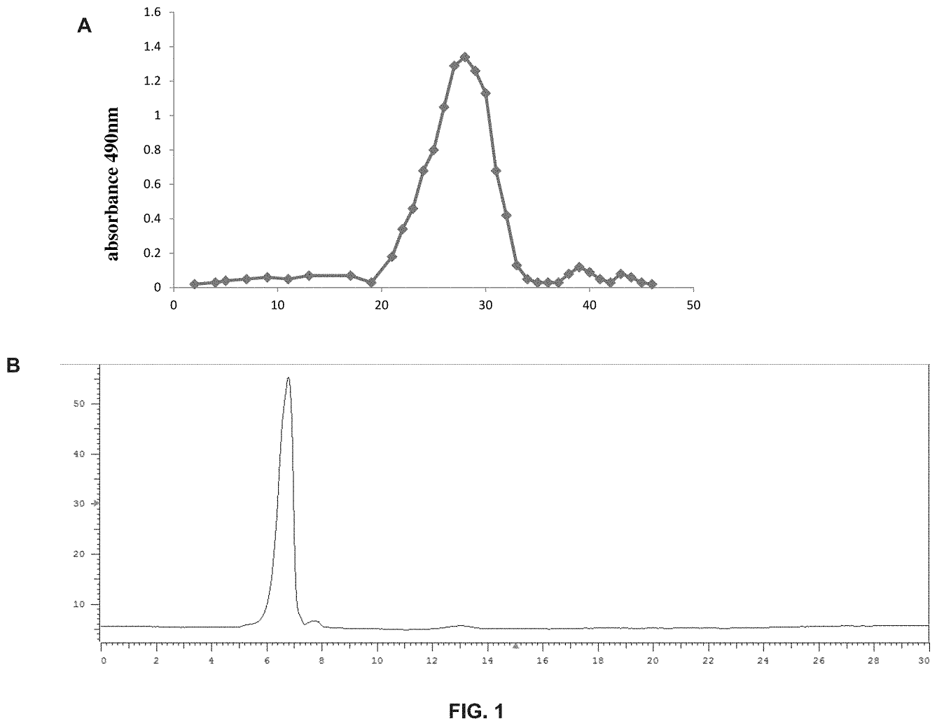

Plot elution curve with optical density value of ESP-B4 (A) HPCE-ELSD profiles of ESP-B4 (B).

### DETAILED DESCRIPTION

The present invention is described in more detail below to facilitate understanding of the present invention.

### Example 1: Extraction and Separation of Homogeneous Polysaccharide ESP-B4 from Ephedra sinica

The crude powder of dried herbaceous stem of Ephedra sinica is 200 kg. It is extracted by reflux with 95% ethanol for 3 times, each time for 3 hours, to remove alkaloids, pigments, polyphenols and oligosaccharides in Ephedra sinica. The residue is decocted with water for 3 times, each time for 3 hours. After cooling, they are filtered and combined with filtrate. The filtrate is recovered by decompression and freeze-dried to obtain 14 kg total polysaccharide of Ephedra sinica. Then, the total polysaccharides are separated by Amberlite FPC3500 and Amberlite IRA-401 anion-cation series resin column and monitored by phenol-sulfuric acid method. First, distilled water is used as eluent, and when no polysaccharide component flows out, 1M NaCl is used for elution. Collect 1 M NaCl eluent, concentrate, dialysis, freeze-drying to obtain elution site Fr.B.

Fr.B is eluted by Cellulose DE-52 column with gradient elution of water, 0.5 M NaCl and 1 M NaCl solution, respectively, at a flow rate of 2 mL/min, followed by phenol-sulfuric acid method. Collect 1 M NaCl main peak eluent, concentrate and freeze-drying. The obtained component is further separated and purified by cellulose DE-52 column using 1 M NaCl solution as eluent. The flow rate was 2 mL/min. Phenol-sulfuric acid method is also used to detect the main elution peaks. The symmetrical main elution peaks is obtained, and the eluents are merged and freeze-dried. The obtained polysaccharide component is named ESP-B4.

The chemical structure feature research of ESP-B4 is as follows:

(1) The polysaccharide of ESP-B4 contains mainly α-glycosidic linkage. Its monosaccharides variety include xylose, arabinose, glucose, rhamnose, mannose, galactose, glucuronic acid and galacturonic acid with the molar percentages 1.5%, 6.8%, 1.5%, 3.0%, 1.5%, 8.3%, 2.3% and 75.2%. ESP-B4 is a typical RG pectin polysaccharide. In addition, there are a few unidentified monosaccharides in constitution of ESP-B4.

(2) ESP-B4 contained the main part of homogalacturonan fragments as “smooth regions”, that is a repeating galacturonic acid interlinkage by →4)-β-D-GalpA-(1→). Rhamnose and galacturonic acid compose of the “nonsmooth region” of main chain with repeated structural segments, that is →4)-β-D-GalpA-(1→2)-α-Rha-(1→). In the “nonsmooth region”, about 70% of the rhamnose residues branch at C-4, and 25.2% galacturonan residues branch at C-3, at which position be substituted by side chains.

(3) The branched structure of ESP-B4 mainly contains arabinan and galactan. Among them, arabinoglycan were (1→5) linkage as the skeleton part, 43.8% of (1→5) Araf branch at C-3. Galactan were (1→3) linkage and (1→4) linkage as the skeleton part, among them, 40.2% of galactan is (1→3) connected, 35.9% of galactan is (1→4) connected, and 70.3% of (1→3) galactan has branch points in its C-6 position, 54.5% of (1→4) galactan has branch points at C-6.

### Example 2: A Tablet Effective Against Respiratory Diseases, Infectious Acute Lung Injury, Respiratory Distress Syndrome, Asthma, Pneumonia, Trachea and Bronchitis

Take an appropriate amount of ESP-B4 prepared in example 1, mixed with diluent, disintegrating agent and other auxiliary materials to form granules, tabletting, coating or thin film coating.

### Example 3: An Oral Liquid Effective Against Respiratory Diseases, Infective Acute Lung Injury, Respiratory Distress Syndrome, Asthma, Pneumonia, Trachea and Bronchitis

Appropriate amount of ESP-B4 prepared in example 1 is added with appropriate water solution, mixing, adding flavoring agents and preservatives, filling, capping and sterilization.

### Example 4: An Infusion Agent Effective Against Respiratory Diseases, Infective Acute Lung Injury, Respiratory Distress Syndrome, Asthma, Pneumonia, Trachea and Bronchitis

Take an appropriate amount of the ESP-B4 prepared in example 1, add a little water for injection to dissolve it, then add an appropriate amount of sodium chloride, dissolve it, then add water for injection to the specified amount, filter, seal and sterilize it.

### Experiment 1: Acute Toxicity Test of ESP-B4

Experimental animals: BALB/c mice (half female and half male), weighing 18-22 g, supplied by drugs safety evaluation center in Heilongjiang University of Chinese Medicine. Feeding temperature: 22.0±1.1° C., humidity: 58.8±11.0%, ventilation times 15 times/h, free feeding and drinking, feed and drinking water compliance with standards.

Methods: Twenty mice, half male and half female, were fed at a volume of 0.4 mL/10 g, ESP-B4 dosage 20 g/kg/d, (up to the maximum dosage and concentration). The mice were fed twice in a day with an interval of 6 hours, and the activity and the number of deaths were observed continuously for 14 days.

Results: LD₅₀ of ESP-B4 was not be detected in mice after oral administration. The maximum dosage of ESP-B4 was 20 g/kg to mice, which was 200 times the dosage of clinical adults. After 14 days of continuous observation, it was found that no mice died, no abnormal changes happened on appearance, physical signs, behavioral activities, mental state, diet, defecation, etc, no abnormal secretions in mouth, eyes, nose, etc.

Conclusion: The acute toxicity test showed that ESP-B4 was safe and non-toxic.

### Experiment 2: Anti-LPS-Induced Acute Lung Injury by ESP-B4

Experimental animals: BALB/c mice, weighing 18-22 g, supplied by drugs safety evaluation center in Heilongjiang University of Chinese Medicine. Feeding temperature: 22.0±1.1° C., humidity: 58.8±11.0%, ventilation times 15 times/h, free feeding and drinking, feed and drinking water compliance with standards.

Methods: Fifty BALB/c mice, half male and half female, after one week of adaptive feeding, were randomly divided into five groups: control group, model group, ESP-B4 low-dosage and high-dosage group and positive control group (dexamethasone group). The mice were anesthetized with 5% chloral hydrate (0.1 mL/20 g) and fixed on the mouse plate apparatus. Blunt separation exposed trachea with tweezers through a small opening at neck, inject 30 μL LPS (5 mg/kg) saline solution into the trachea, rotate the plate for 1 min, and then suture the wound to establish acute lung injury model. The dosage of dexamethasone was 50 mg/kg, ESP-B4 low-dosage group was 50 mg/kg and ESP-B4 high-dosage group was 100 mg/kg in the. Each group intraperitoneal injection. The control group was given the same volume of normal saline. The mice were given continuous administration for 7 days. After the last administration for 2 hours, the mice were executed by cervical dislocation. The left lung tissues were quickly removed and grounded on ice to obtain homogenate to detect levels of the inflammatory factors TNF-α, IL-6, IL-8 and Tp (Total protein), as follows:

Statistical Methods: All data were expressed as mean±SD, and t-test was used for comparison between groups.

TABLE 1
Effects of ESP-B4 on LPS-induced acute lung injury in mice

| Group | TNF-α (pg/mL) | IL-6 (pg/mL) | IL-8 (pg/mL) | Tp (μg/mL) |
|---|---|---|---|---|
| Control group | 793.46 ± 50.93 | 331.94 ± 30.20 | 26.21 ± 5.87 | 164.44 ± 27.44 |
| Model group | 1233.49 ± 27.91^{▴▴} | 905.52 ± 43.31^{▴▴} | 153.23 ± 11.24^{▴▴} | 304.78 ± 18.96^{▴▴} |
| ESP-B4 low-dose group | 1189.58 ± 19.38 | 423.46 ± 20.18** | 133.84 ± 12.69 | 282.24 ± 19.89 |
| ESP-B4 high-dose group | 977.89 ± 85.92* | 366.18 ± 35.41** | 84.84 ± 10.31** | 211.04 ± 15.61* |
| Dexamethasone group | 861.41 ± 71.04** | 255.18 ± 35.63** | 52.7 ± 9.99** | 202.40 ± 16.54** |

Compared to the control group,
^{▴}P < 0.05;
^{▴▴}P < 0.01,
Compared to model group
*P < 0.05,
**P < 0.01.

Results: The levels of inflammatory factors TNF-α, IL-6, IL-8 and Tp in lung tissue of acute lung injury model group were significantly higher than control group (P<0.01), indicating that the model was successful. Compared with the model group, the levels of TNF-α, IL-6, IL-8 and Tp in the ESP-B4 high-dose group and dexamethasone group of the present invention are significantly reduced. Among them, the levels of Tp and dexamethasone groups are roughly the same. The levels of TNF-α, IL-8 and Tp in the ESP-B4 low-dose group are also reduced, and the differences of IL-6 are significant.

Conclusion: ESP-B4 showed an obvious dose-dependent therapeutic effect on LPS-induced acute lung injury in mice.

### Experiment 3: Anti-E. coli-Induced Pneumonia Effect of ESP-B4

Experimental Animals: BALB/c mice, weighing 18-22 g, supplied by drugs safety evaluation center in Heilongjiang University of Chinese Medicine. Feeding temperature: 22.0±1.1° C., humidity: 58.8±11.0%, ventilation times 15 times/h, free feeding and drinking, feed and drinking water compliance with standards.

Methods: Fifty BALB/c mice were randomly divided into five groups: control group, model group, ESP-B4 low-dose group, ESP-B4 high-dose group and gentamicin sulfate positive control group. The pneumonia model was established by injection of E. coli. The mice were anesthetized with 5% chloral hydrate (0.1 ml/20 g) and fixed on the mouse plate apparatus. Blunt separation exposed trachea with tweezers through a small opening at neck, inject 30 μL E. coli. (2×10⁸ CFU/mL) into the trachea, rotate the plate for 1 min, and then suture the wound to establish pneumonia model. Each group of mice was administered intragastrically. The dosage of gentamicin sulfate group was 100 mg/kg, ESP-B4 low-dose group was 75 mg/kg, and ESP-B4 high-dose group was 150 mg/kg. After 7 days of continuous intragastric administration, the mice were executed by cervical dislocation after 2 hours of the last administration. The left lung tissue was quickly removed and grounded on ice to obtain homogenate to detect levels of the inflammatory factors TNF-α, IL-6, IL-8 and Tp, as follows:

Statistical Methods: All data were expressed as mean±SD, and t-test was used for comparison between groups.

TABLE 2
of ESP-B4 on Escherichia coli-induced pneumonia in mice

| Group | TNF-α (pg/mL) | IL-6 (pg/mL) | IL-8 (pg/mL) | Tp (μg/mL) |
|---|---|---|---|---|
| Control group | 803.23 ± 75.82 | 397.55 ± 32.22 | 30.06 ± 4.85 | 151.15 ± 28.06 |
| Model group | 1405.65 ± 93.03^{▴▴} | 953.36 ± 55.25^{▴▴} | 175.65 ± 12.02^{▴▴} | 327.25 ± 20.12^{▴▴} |
| ESP-B4 low dose group | 1112.58 ± 88.55* | 488.56 ± 40.02** | 138.38 ± 13.99* | 292.12 ± 22.26 |
| ESP-B4 high dose group | 902.18 ± 74.22** | 387.36 ± 39.39** | 88.69 ± 11.07** | 223.26 ± 15.52* |
| Gentamycin Sulfate group | 834.10 ± 70.10** | 285.16 ± 32.71** | 49.36 ± 10.01** | 197.05 ± 14.20** |

Compared to control group,
^{▴}P < 0.05;
^{▴▴}P < 0.01,
Compared to model group
*P < 0.05,
**P < 0.01.

Results: The levels of inflammatory factors TNF-α, IL-6, IL-8 and Tp in lung tissue of pneumonia model group were significantly higher than control group (P<0.01), indicating that the model was successful. Compared with the model group, TNF-α, IL-6 and IL-8 in the ESP-B4 low-dose and high-dose group and gentamicin sulfate group were significantly decreased (P<0.05) and showed a dose-effect relationship, while the Tp content in the high-dose group and gentamicin sulfate group was significantly decreased (P<0.05).

Conclusion: ESP-B4 has obvious therapeutic effect on E. coli-induced pneumonia in mice.

### Experiment 4: Anti-H5NI-Induced Acute Lung Injury by ESP-B4

Laboratory Animals: BALB/c mice, weighing 18-22 g, supplied by drugs safety evaluation center in Heilongjiang University of Chinese Medicine. Feeding temperature: 22.0±1.1° C., humidity: 58.8±11.0%, ventilation times 15 times/h, free feeding and drinking, feed and drinking water compliance with standards.

Methods: After a week of adaptive feeding, fifty BALB/c mice, half male and half female were randomly divided into five groups: control group, model group, ESP-B4 low-dose group, ESP-B4 high-dose group and oseltamivir phosphate positive drug group. Acute lung injury was induced by intratracheal injection of H5N1. The mice were anesthetized with 5% chloral hydrate (0.1 ml/20 g) and fixed on the mouse plate apparatus. Blunt separation exposed trachea with tweezers through a small opening at neck, inject 30 μL H5N1 (5×10⁴LD₅₀/mL) into the trachea, rotate the plate for 1 min, and then suture the wound to establish pneumonia model. Each group of mice was administered intragastrically. The dosage of oseltamivir phosphate group was 50 mg/kg, ESP-B4 low-dose group was 50 mg/kg, and ESP-B4 high-dose group was 100 mg/kg. After 7 days of continuous administration, the mice were executed by cervical dislocation after 2 hours of the last administration. The left lung tissues were quickly removed and grounded on ice to obtain homogenate to detect levels of the inflammatory factors TNF-α, IL-6, IL-8 and Tp, as follows:

Statistical Methods: All data were expressed as mean±SD, and t-test was used for comparison between groups.

TABLE 3
Effects of ESP-B4 on H5NI-induced acute lung injury in mice

| | | | | Total protein |
|---|---|---|---|---|
| Group | TNF-α (pg/mL) | IL-6 (pg/mL) | IL-8 (pg/mL) | (μg/mL) |
| Control group | 745.41 ± 48.81 | 368.56 ± 35.53 | 31.65 ± 7.85 | 134.64 ± 17.55 |
| Model group | 1389.52 ± 38.99^{▴▴} | 1003.35 ± 60.15^{▴▴} | 201.85 ± 20.77^{▴▴} | 289.89 ± 16.46^{▴▴} |
| ESP-B4 low dose group | 1107.12 ± 89.29 | 565.63 ± 44.42** | 127.51 ± 15.84* | 244.34 ± 20.41 |
| ESP-B4 high dose group | 988.81 ± 70.23* | 402.21 ± 40.15** | 91.52 ± 11.77** | 207.51 ± 17.88* |
| Oseltamivir phosphate | 901.55 ± 68.58** | 328.21 ± 34.25** | 45.64 ± 7.75** | 176.62 ± 17.69** |
| group | | | | |

Compared to the control group,
^{▴}P < 0.05;
^{▴▴}P < 0.01,
Compared to model group
*P < 0.05,
**P < 0.01.

Results: The levels of inflammatory factors TNF-α, IL-6, IL-8 and Tp in lung tissue of acute lung injury model group were significantly higher than those of control group (P<0.01), indicating that the model was successful. Compared with the model group, TNF-α, IL-6, IL-8 and Tp in the ESP-B4 high dose group and oseltamivir phosphate group are significantly reduced, and there are significant differences (P<0.05). Compared with the model group, TNF-α and Tp in the ESP-B4 low dose group are also decreased, and IL-6 and IL-8 are significantly different.

Conclusion: ESP-B4 has obvious therapeutic effect on H5NI-induced acute lung injury in mice.

The preferred embodiments of the present invention are described above, but they are not intended to define the present invention. Technicians in the research field may make improvements and changes to the implementation scheme disclosed herein without departing from the scope and spirit of the present invention.

What is claimed is:

1. A homogeneous polysaccharide ESP-B4 from Ephedra sinica for treating a respiratory disease, where in the respiratory disease includes acute lung injury and respiratory distress syndrome.

2. The polysaccharide ESP-B4 from Ephedra sinica according to claim 1, wherein the polysaccharide ESP-B4 is isolated and purified from the stems of Ephedra sinica Stapf, Ephedra intermedia Schrenk et C. A. Mey., or Ephedra equisetina Bge.

3. The polysaccharide ESP-B4 from Ephedra sinica according to claim 1, wherein the polysaccharide ESP-B4 is acid heteropolysaccharide, the monosaccharide is composed of xylose, arabinose, glucose, rhamnose, mannose, galactose, glucuronic acid and galacturonic acid with a molar ratio of 1.0:4.5:1.0:2.0:5.5:1.5:50, and the molar percentage of galacturonic acid is 75.2%, the molecular weight of ESP-B4 is 2.37×10⁷ Da.

4. A method for preparing the polysaccharide ESP-B4 from Ephedra sinica according to claim 1, comprising the following steps:
Step (1): preparing total polysaccharides from Ephedra sinica by water extraction and alcohol precipitation;
Step (2): separating the total polysaccharides by series chromatography of Anion and Cation resin column; and
Step (3): separating polysaccharide ESP-B4 by separation of cellulose and sepharose chromatography column through ion exchange, adsorption and molecular sieve m.

5. The method according to claim 4, wherein:
in step (1), the water extraction is performed by extracting 2-3 times with 5-10 times hot water, and the ethanol concentration of alcohol precipitation is 75%-87%;
in step (2), anion and cation resin packing are preferably Amberlite FPC3500 and IRA-401;
in step (3), the Cellulose column filler and DEAE-Sepharose F.F are preferably used; and
the chromatographic packing materials used in step (2) and step (3) are not restricted to the above selection, regardless of the packing choice, the purity of the prepared ESP-B4 is more than 85%.

6. A pharmaceutical composition for treating acute lung injury and respiratory distress syndrome, comprising the polysaccharide ESP-B4 from Ephedra sinica and a pharmaceutically acceptable carrier.

7. The pharmaceutical composition according to claim 6, wherein the pharmaceutical composition is in the dosage form of oral preparation or parenteral preparation, the oral preparation is selected from tablet, medicinal granules, capsule, oral liquid or pill, the parenteral preparation is selected from injection or infusion solution, the capsule is selected from hard capsule and soft capsule, the pill is dropping pill, the injection is selected from injection solution, freeze-dried powder injection, water injection, the infusion solution is large volume injection.

8. A method for treating acute lung injury comprising administering to a subject in need thereof a therapeutically effective amount of the polysaccharide ESP-B4 according to claim 1.

9. The method according to claim 8, wherein the acute lung injury is selected from infectious acute lung injury and respiratory distress syndrome caused by infectious acute lung injury deterioration, the infectious acute lung injury is selected from major infectious acute lung injury caused by SARS virus, influenza A virus, avian influenza virus, as well as pulmonary infection caused by Escherichia coli, Pseudomonas aeruginosa, Pneumococcus, Staphylococcus aureus, Klebsiella pneumoniae, influenza virus, Chlamydia and Mycoplasma infection.
